(19) United States
(12) Patent Application Publication
(10) Pub. No.: US 2006/0057674 A1
(43) Pub. Date: Mar. 16, 2006

(54) Translocating enzyme as a selection marker

(76) Inventors: Maren Hintz, Dusseldorf (DE); Roland Freudl, Duren (DE); Jorg Feesche, Erkrath (DE); Roland Breves, Mettmann (DE)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/216,333

(22) Filed: Aug. 31, 2005

Related U.S. Application Data
(63) Continuation of application No. PCT/EP04/01949, filed on Feb. 27, 2004.

(30) Foreign Application Priority Data
Mar. 4, 2003 (DE) 103 09 557.8

(51) Int. Cl. C12P 21/06; C07H 21/04; C12N 9/00; C12N 15/74

(52) U.S. Cl. 435/69.1; 435/471; 435/252.3; 536/23.2; 435/183

(57) ABSTRACT

The subject of the present invention is a selection system for microorganisms, which is based on the inactivation of an essential translocating enzyme and the curing of this inactivation by means of an indentically acting factor which is made available to the cells concerned by means of a vector. One important area of application for this system is includes processses for protein production by culturing cells of a microorganism strain that are characterized by this selection system, particularly such that the transgene of interest is located on the same vector performing the cure. Appropriate microorganisms, possible uses for genes of translocation enzymes and vectors are likewise presented, including in particular the use of the gene secA from gram-negative or gram-positive bacteria such as B. licheniformis.

### CROSS REFERENCE TO RELATED APPLICATIONS

This application is a §365 (c) continuation application of PCT/EP2004/001949 filed 27 Feb. 2004, which in turn claims priority to DE Application 103 09 557.8 filed 4 Mar. 2003, each of the foregoing applications is incorporated herein by reference.

### FIELD OF THE INVENTION

The present invention relates to a selection system for microorganisms, which is based on the inactivation of an essential translocating enzyme and the curing of this inactivation by means of an identically acting factor which is made available to the cells concerned by means of a vector.

### BACKGROUND OF THE INVENTION

Fermentation of microorganisms is frequently employed to produce large quantities of desirable proteins, particularly enzymes useful for industrial application. While many microorganisms naturally form the proteins of interest, genetically modified producer strains are increasingly gaining importance. Genetic engineering processes for enhancing protein production have long been established in the prior art. Typically, genes encoding the proteins of interest are incorporated into host cells as transgenes, transcribed and translated, and optionally secreted into the periplasma, or the surrounding medium. Following production and/or secretion such proteins are readily obtainable from the cells concerned or the culture supernatants.

Protein production on an industrial scale typically takes advantage of the natural abilities of microorganisms which produce and/or secrete the protein of interest. Basically, the bacterial systems selected for protein production are those which are inexpensive and amenable to fermentation, capable of producing large quantities of protein product and facilitate correct folding, modification etc. of the protein to be produced. The latter is all the more probable with increasing relationship with the organism originally producing the protein of interest. Host cells particularly established for this purpose are gram-negative bacteria, such as, for example, Escherichia coli or Klebsiella, or gram-positive bacteria, such as, for example, species of the genera Staphylococcus or Bacillus.

The economy of a biotechnological process is critically dependent on the achievable yield of protein. This yield is determined by several factors, e.g., the expression system employed; the growth parameters utilized including the fermentation parameters and substrates supplied in the media. By optimization of the expression system and of the fermentation process, the achievable yield of protein production can be markedly increased.

Two different genetic approaches are typically employed to enhance production of proteins of interest. In one approach, the gene for the protein to be produced is integrated into the chromosome of the host organism. Constructs of this type are very stable to the presence of an additional marker gene without selection (see below). The disadvantage is that only one copy of the gene is present in the host and the integration of further copies to increase the product formation rate by means of a gene dose effect is quite complicated. Prior art describing this approach is briefly illustrated below.

European patent EP 284126 B1 solves the problem of stable multiple integration in that a number of gene copies are incorporated into the cell, which contain the endogenous and essential chromosomal DNA sections lying in between.

Another solution to stable multiple integration is disclosed in patent application WO 99/41358 A1. Two copies of the gene of interest are integrated in opposite transcription directions and are separated from one another by a nonessential DNA sequence in order to prevent homologous recombination of the two copies.

Patent application DD 277467 A1 discloses a process for the production of extracellular enzymes which is based on the stable, advantageously multiple, integration of the genes coding for the enzyme of interest into the bacterial chromosome. The integration takes place via homologous recombination. Successful integration events are monitored by including an erythromycin gene on the plasmid employed which is inactivated upon successful integration.

According to specification of DE 4231764 A1, integration into the chromosome can take place via single or double crossing-over events using constructs that include the gene for thymidylate synthetase. Inclusion of thymidylate synthetase facilitates control and monitoring of this process, e.g., a single crossing-over event results in retention of thy activity, whereas enzyme activity is lost upon double crossing-over. Loss of enzyme activity gives rise to an auxotrophy phenotype. Resistance to the antibiotic trimethroprim results for a single crossing-over event whereas a double crossing-over event confers sensitivity to this antibiotic.

In application WO 96/23073 A1, a transposon-based system for integration of multiple copies of the gene of interest into the bacterial chromosome is disclosed. In this system, the marker gene of the plasmid is deleted by the integration and the strains contained are thus free of a resistance marker. Also, according to this specification, a marker is only needed for the control of the construction of the bacterial strain concerned.

A system for increasing the copy number of certain transgenes integrated into a bacterial chromosome is also disclosed in application WO 01/90393 A1.

The second approach for the construction of producer autonomously replicating element, (e.g., a plasmid), followed by introduction of the element into a host organism. The customarily high number of plasmid copies per cell provides advantages via a gene dose effect. One drawback to this approach is that selection pressure must be continuously applied during culture to maintain the plasmids in the cells. Typically, such plasmids carry antibiotic resistance genes. The addition of antibiotics to the culture medium selects for those cells which carry the plasmid such that only the cells which possess the plasmids (which also carry the transgene) in adequate number are able to grow.

Recently, the application of antibiotic resistance selection is increasingly running into criticism. On the one hand, the application of antibiotics is quite expensive, particularly in those cases where resistance is based on an enzyme degrading the antibiotic. In this instance, the substance concerned must be added during the entire culture period. On the other hand, widespread use of antibiotics, in particular in other technical fields, contributes to the spread of the resistance genes to other strains, which include pathological strains. In the field of medical hygiene, and in particular in the treatment of infectious diseases, such widespread use of antibiotics has given rise to ‘multiresistant human-pathogenic strains’ which provide clinical challenges to the physician.

Therefore, to a great extent, regress is made to the systems illustrated above for the stable integration of genes into the chromosome of the producer cells, because these are stable without application of continuous selection pressure. However, the strains concerned, as mentioned above, can only be prepared with great expense. It is quicker and more convenient in biotechnological practice to incorporate newly found or modified genes encoding a protein of interest into a plasmid with selection markers, introducing the plasmid into host interest.

In the prior art, antibiotic-free selection systems have also been developed. For instance, in the publication “Transposon vectors containing non-antibiotic resistance selection markers for cloning and stable chromosomal insertion of foreign genes in gram-negative bacteria” by Herrero et al. (1990), in J. Bacteriol., Volume 172, pages 6557-6567, resistance to herbicides and heavy metals as selection markers are described. Application of these compounds, however, presents the same concerns as those discussed above with regard to widespread antibiotic use.

Selection via auxotrophy, e.g., via a specific metabolic defect which makes the cells concerned dependent on the supply of certain metabolic products, functions similarly in principle to an antibiotic selection. Auxotrophic strains receive, coupled with the transgene of interest, a plasmid which contains nucleic acids encoding the defective or deleted molecule, thereby curing this auxotrophy. In the case of loss, under appropriate culture conditions cells would simultaneously lose their viability, such that the desired selection of the auxotrophic producer strains occurs. For instance, in the publication “Gene cloning in lactic streptococci” by de Vos in Netherlands Milk and Dairy Journal, Volume 40, (1986), page 141-154, reference is made, for example, on p. 148 to various selection markers developed from the metabolism of lacto-streptococci; among these are those from lactose metabolism, copper resistance and resistance genes to various bacteriocins of lacto-streptococci. Patent EP 284126 B1, which relates to the stable integration of genes of interest into the bacterial chromosome (see above) summarizes the systems auxotrophy, resistance to biocides and resistance to virus infections possible for selection on p. 7 under the term “Survival selection”. Examples of auxotrophy selection markers mentioned include the metabolic genes leu, his, trp “or similar” which clearly refers to additional amino acid synthesis pathways.

In practice, the application of auxotrophic selection has been problematic since industrial fermentation media include almost all necessary substrates in adequate amounts. Thus, cells can compensate for the shortage of the synthesis of a certain compound by taking up this same compound from the nutrient medium.

Thymidine is present in industrial fermentation media in trace amounts and therefore must be formed from the proliferating, and thus DNA-synthesizing organisms by means of a thymidylate synthase. Thus, application EP 251579 A2 offers the solution of employing as host strains those which are deficient with respect to the gene for thymidylate synthase which is essential for nucleotide metabolism. By means of a vector, it is accordingly possible to make available the gene for precisely this function (thyA from Escherichia coli K12) and to cure the gene defect. If this vector additionally carries the gene for the protein of interest, an antibiotic-like selection of the producer cells occurs.

In summary, while the prior art discloses a variety of approaches for the biotechnological production of proteins and the expression of genes of interest (e.g., chromosomal integration, and antibiotic selection of plasmids containing transgenes and selectable markers), to date, no practical alternatives to these approaches exist, particularly systems which are less complicated than chromosomal integration and at the same time manage without selection by means of an expensive or ecologically questionable compound. Selection by means of auxotrophy markers has up to now led only to limited results due to the complex nutrient media generally customary in industry.

### SUMMARY OF THE INVENTION

Thus, an object of the invention is to provide a new selection system which is as comparatively simple to handle as selection via an antibiotic without employing expensive and, under certain circumstances, environmentally harmful substances. The system of the invention is amenable to use on an industrial scale and is not based on an essential gene whose absence in industrial media can be compensated for by contaminants.

This object is achieved according to the invention by processes for the selection of a microorganism, comprising,
(a) inactivating an endogenous gene which encodes an essential translocation activity in said microorganism; and
(b) introducing a vector into said microorganism which encodes a protein comprising said essential translocation activity, thereby curing the inactivation of said translocation activity, the vector optionally further comprising a transgene. In a preferred embodiment, the vector of b) comprises a transgene encoding a protein of interest.

In one aspect, the essential translocation activity is expressed from a nucleic acid which encodes a factor selected from the group consisting of SecA, SecY, SecE, SecD, SecF, signal peptidase, b-SRP (Ffh or Ffs/Scr), FtsY/Srb, PrsA or YajC. More preferably, the nucleic acid encodes one subunit of the preprotein translocase selected from the group consisting of SecA, SecY, SecE, SecD or SecF. In preferred embodiments, the subunit is SecA encoded by a nucleic acid selected from the group consisting of SEQ ID NO: 1, SEQ ID NO: 3 and SEQ ID NO: 5.

In a further aspect of the invention, the inactivation according a) results in a deletion of the endogenous nucleic acid sequence encoding the essential translocation activity, such that recombination between the curing vector of b) and the homologous chromosomal be effected by a deletion vector which comprises an externally regulatable replication origin.

It is preferred that the vector according to b) comprises a plasmid which replicates autonomously in the microorganism. Preferably, the plasmid is a multiple copy number plasmid.

Also encompassed by the present invention is a process for the preparation and isolation of a protein of interest comprising selecting the microorganism for production by
(a) inactivating an endogenous gene which encodes an essential translocation activity in said microorganism;
(b) introducing a vector into said microorganism which encodes a protein comprising said essential translocation activity, thereby curing the inactivation of said translocation activity, said vector comprising a transgene encoding said protein protein of interest under conditions where said protein is produced; and
c) isolating said protein of interest.

In a further aspect, microorganisms, obtainable by the selection process as disclosed herein are included within the scope of the invention.

Finally, the vectors which effect the curing of step b) are also provided.

### BRIEF DESCRIPTION OF THE DRAWINGS

FIG. 1: Schematic representation of the translation/translocation apparatus of gram-positive bacteria Analogously according to van Wely, K. H., Swaving, J., Freudl, R., Driessen, A. J. (2001); “Translocation of proteins across the cell envelope of Gram-positive bacteria”, FEMS Microbiol Rev. 2001, 25(4), pp. 437-54).

FIG. 2: Gene locus of SecA in B. subtilis It is recognized that the gene prfB also lies in the SecA region and a related mRNA is formed, so that it is also possible to speak of a SecA/prfB operon.

FIG. 3: Restriction map of the gene locus orf189/SecA/prfB in B. licheniformis As shown in example 1, the gene prfB and an orf on a fragment about 5.5 kB in size are located in the immediate vicinity of SecA, which are readily obtainable from the genomic DNA of B. licheniformis using restriction digest with MunI.

FIG. 4: Preparation of a plasmid having a SecA gene and a subtilisin gene As described in example 2, SecA was amplified by means of PCR and cloned into a vector which contains alkaline protease from B. lentus as the exemplary transgene.

FIG. 5: Regions of SecA (up- and downstream) amplified by means of PCR Amplification of the up- and downstream regions of SecA using the restriction cleavage sites selected for cloning as described in example 3. The 3′ end of orf189 is amplified using its own terminator and the SecA promoter lying downstream, so that after SecA deletion the prfB can be transcribed directly from the SecA promoter. The sections orf189‘and prfB’ derived in each case comprise 502 bp or 546 bp.

FIG. 6: Construction of the deletion plasmid pEorfprfB The regions amplified by means of PCR were cloned into E. coli, excised again by means of XbaI and EcoRV and subsequently ligated into the restriction cleavage sites XbaI and AccI in the vector pE194.

FIG. 7: Plasmid stability in the transformants B. licheniformis (SecA) pCB56C (control) and B. licheniformis (ΔSecA) pCB56CSecA The fraction of the clones having protease activity is in each case applied, as described in example 4, after an appropriate number of days.

Squares: B. licheniformis (ΔSecA) pCB56CSecA
Triangles: B. licheniformis (SecA) pCB56C (control)

### DETAILED DESCRIPTION OF THE INVENTION

Surprisingly, it has been discovered that the essential protein factors which mediate protein translocation are suitable for use as selection markers. In accordance with the present invention, a gene encoding an essential protein involved in protein translocation is used as the selection marker. Accordingly, absence or inactivation of this gene is lethal and thus an antibiotic-like selection of microorganisms is possible. Advantageously, this selection system can be practiced without additives (such as, for example, the antibiotics discussed above) and in principle functions independently of the composition of the nutrient media. Recombinant molecular biological techniques are employed to modify the translocation machinery of the microorganism in which the protein of interest is to be produced. Such techniques are described in the following examples.

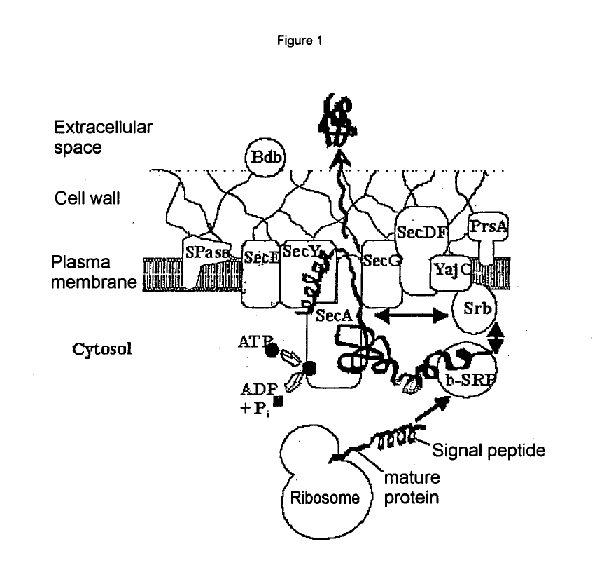

The process of translocation involves the secretion of proteins formed by bacteria into the periplasma (in the case of gram-negative bacteria), or the surrounding medium (both in the case of gram-negative and in the case of gram-positive bacteria). The process is described, for example, in A. J. Driessen (1994): “How proteins cross the bacterial cytoplasmic membrane” in J. Membr. Biol., 142 (2), pp. 145-59. The secretion apparatus consists of a series of diverse, mainly membrane-associated proteins, which are shown in FIG. 1 of the present application. These include, in particular, the proteins SecA, SecD, SecF (together as the complex SecDF), E, G and Y well characterized, for example, for Bacillus subtilis (van Wely, K. H., Swaving, J., Freudl, R., Driessen, A. J. (2001): “Translocation of proteins across the cell envelope of Gram-positive bacteria”, FEMS Microbiol Rev. 2001, 25(4), pp. 437-54). Further factors to be considered part of this system are YajC, which likewise comes into direct contact with the Sec complex, and the factors Bdb (Dsb), SPase (for “signal peptidase”), PrsA and b-SRP (Ffh, Ffs/Scr, SRP-RNA) which are also shown in FIG. 1.

The last-mentioned factor is a bacterial factor, which in theory functions as an SRP (signal recognition particle) comparable to that described originally in eukaryotes. Ffh, a subunit of this particle, which is characterized both from B. Subtilis and from E. coli. Another subunit of b-SRP is called Scr in B. subtilis and Ffs in E. coli. Furthermore, an RNA (SRP-RNA) is part of the functional b-SRP complex. A further factor functionally associated with this particle is referred to as Srb in E. coli and FtsY in B. subtilis. This molecule corresponds functionally to the eukaryotic docking protein.

Additionally, PrfB (peptide chain release factor B; also RF2) is also to be included. This molecule functions in translation termination during protein synthesis in both gram-positive and in gram-negative bacteria and facilitates detachment of the ready-translated proteins from the ribosome. The relationship to the translocation presented above is only indirectly afforded in that the gene prfB in many bacteria is transcribed simultaneously with the gene for the factor SecA. There is thus a regulatory relationship.

The prerequisite for translocation is that the proteins to be discharged have a signal peptide N-terminally (Park, S., Liu, G., Topping, T. B., Cover, W. H., Randall, L. L. (1988): “Modulation of folding pathways of exported proteins by the leader sequence”, Science, 239, pp. 1033-5). This applies both to extracellular proteins and to membrane proteins.

Following translation of mRNA on the ribosome, the newly synthesized peptide chain remains in an unfolded state and are transported to the membrane via the action of cytoplasmic proteins having a chaperone function. The transport of the peptide through the membrane is then catalyzed via the consumption of ATP (Mitchell, C., Oliver, D. (1993): Two distinct ATP-binding domains are needed to promote protein export by Escherichia coli SecA ATPase”, Mol. Microbiol., 10(3), pp. 483-97). SecA functions as an energy-supplying component (ATPase) of the multienzyme complex translocase. After crossing the membrane, the signal peptide is cleaved by a signal peptidase and the extra-cellular protein is detached from the membrane. In the case of gram-positive bacteria, the discharge of the exoproteins occurs directly into the surrounding medium. In the case of gram-negative bacteria, the proteins are subsequently found, as a rule, in the periplasma and further modifications are needed in order to achieve their release into the surrounding medium.

The preprotein translocase consists of the subunits SecA, SecY, SecE, SecD, SecF (SecDF) and SecG. As the ATPase controlling this process, the factor SecA is essential for translocation. Accordingly, the preferred embodiments of the system of the present invention comprises the use of these factors (see below).

Table 1 below classifies the factors set forth as essential in one of the two model organisms Escherichia coli (gram-negative) and Bacillus subtilis (gram-positive). Any factor designated as essential is suitable for use in the selection system of the invention. Use of homologs of the indicated proteins in other species of gram-negative and gram-positive bacteria is also encompassed within the scope of the invention.

TABLE 1
Protein factors which modulate protein translocation in
gram-negative and gram-positive bacteria, classified according
to whether they are essential in these organisms.

| | E. coli | B. subtilis |
|---|---|---|
| SecA | essential | essential |
| SecY | essential | essential |
| SecE | essential | essential |
| SecG | nonessential | nonessential |
| | (cold-sensitive | (cold-sensitive |
| | phenotype) | phenotype with |
| | | overproduction of |
| | | export proteins) |
| SecD, SecF | essential | nonessential |
| (SecDF) | | (cold-sensitive |
| | | phenotype) |
| Signal | essential | nonessential, since |
| peptidase | | present in |
| | | redundant form |
| b-SRP (Ffh; | essential | essential |
| Ffs/Scr; SRP- | | |
| RNA) | | |
| FtsY/Srb | essential | essential |
| PrsA | not present | essential |
| Bdb/Dsb | nonessential | nonessential |
| YajC | essential | not known whether |
| | | essential, but |
| | | present in |
| | | redundant form |

According to the invention, the following can thus be selected in gram-negative bacteria, in particular in coliform bacteria, very particularly in E. Coli, via the inactivation of the following translocating enzymes or their associated genes: SecA, SecY, SecE, SecD, SecF, signal peptidase, b-SRP (Ffh or FfS), Srb or YajC.

According to the invention, the following can thus be selected in gram-positive bacteria, in particular in Bacillus, very particularly in B. subtilis, via the inactivation of the following translocating enzymes or their associated genes: SecA, SecY, SecE, b-SRP (Ffh or Scr), FtsY or PrsA.

The “or” connection in these lists is not to be understood exclusively. Technically, it should be possible also to switch off a number of the associated genes simultaneously. According to the invention, however, it is sufficient to select only one for this.

In each case, individual sequences of the associated genes are obtainable, for example, from the following generally accessible data banks: GenBank (National Center For Biotechnology Information NCBI, National Institutes of Health, Bethesda, Md., USA; www3.ncbi.nlm.nih.gov); EMBL European Bio-informatics Institute (EBI) in Cambridge, Great Britain (www.ebi.ac.uk); Swiss-Prot (Geneva Bio-informatics (GeneBio) S. A., Geneva, Switzerland; www.genebio.com/sprot.html); “Subtilist” or “Colibri” of the Pasteur Institute, 25, 28 rue du Docteur Roux, 75724 Paris CEDEX 15, France for genes and factors from B. subtilis or E. coli (genolist.pasteur.fr/SubtiList/ or genolist.pasteur.fr/Colibri/). Furthermore, other databases are available which can be reached via cross-referencing the data banks mentioned above. According to the invention, it is in each case only necessary to identify and to use appropriately a single essential gene of the translocation apparatus in the strain intended for culturing.

The sequences for the factor SecA from various microorganisms indicated in the sequence listing for the present application provide a further starting point. These can be used either directly (see below: preferred embodiments) or be employed in order to identify the homolog concerned in a gene bank which has been designed beforehand for the microorganism of interest.

Preferably, these translocating enzymes or factors are wild-type molecules. However, variants thereof may be prepared which have function comparable to the wild-type enzyme in the translocation apparatus. Accordingly selection systems using such homologs are also included in the scope of the invention.

In order to achieve the object of the invention, strains can be cultured and assessed to identify those factors which are essential to translocation. This is possible in a simple manner, for example by removing one of these known genes from a strain which is as closely related as possible (for example in a likewise gram-negative or gram-positive bacterium) or by recombinantly producing a knock-out vector specific for the molecule using sequence information obtainable from generally accessible data bases. A procedure of this type is generally known to the person skilled in the art. If the transformation with this vector and a subsequent (preferably initiated separately from the transformation) homologous recombination of this gene into the genome of the host cell has a lethal effect, the gene is to be regarded as essential. This essential gene can now be employed according to the invention as a selection marker and in particular according to the model of the examples of the present application.

An inactivation according to step (a) of the present method is performed, for example, by means of homologous recombination of an inactivated gene copy, which has been introduced into a cell of the microorganism strain of interest, for example by transformation with an appropriate vector. Methods for this are known per se. As a result of the recombination event, the chromosomal copy of the gene is completely or partially deleted and thus incapable of function. This can be carried out, for example, by means of the same gene with which the test for lethality has been carried out beforehand. Preferably, however, the endogenous homolog, provided it is known or can be isolated with justifiable expenditure, is employed in order to achieve a high success rate for the recombination. Whether the inactivation is successful is decisive for the accomplishment of the invention.

In one embodiment, plasmid vectors are employed which possess a temperature-sensitive replication origin and into which the homologous DNA regions of the gene targeted for deletion have additionally been inserted (deletion vector). A reversible inactivation, for example, would also be conceivable, for example by means of integration of a mobile genetic element, for example a transposon, into the target gene.

In this context, in each case feature (b) is to be taken into account, namely that even before this recombination or inactivation event, or at the latest simultaneously, an intact copy of the gene selected for the selection according to the invention is prepared in the cell concerned, because the cell would otherwise not survive the inactivation. According to the invention, the resulting defect is compensated by means of a vector, that is to say the vector cures the inactivation. In this context, as mentioned above, the genes endogenously present in the host cells and deleted according to (a) are preferably used. However, functionally identical genes from other organisms, preferably related strains, can also be employed provided they are able to cure the defect concerned. Thus it is possible, for example, to cure the defect of SecA of a B. subtilis by provision of a SecA gene from Staphylococcus carnosus.

It would also be conceivable that by means of the vector another genetic element abolishing the first defect is brought into the cell, for example the gene of a factor which is in principle identical functionally, but modified by mutation.

In this cell, a situation thus prevails in which a lethal defect is compensated by means of a separate genetic element. A loss of this separate genetic element would in turn be lethal, so that such a cell is forced in the case of any cell division to pass on this element to the subsequent generation.

Feature (b) indicates that the vector which cures the defect optionally contains a transgene encoding the desired protein of interest. Preferably the vector of b) does contain a transgene (see below). In this embodiment, the vector compensating the gene defect carries the transgene encoding the protein of interest, which can then be isolated by means of the process according to the invention.

An endogenous selection pressure to a certain extent prevails, without the addition of another compound, for example of a heavy metal or of an antibiotic, being necessary from outside, that is to say via the nutrient medium, in order to prevent the loss of the vector having the transgene. On the other hand, the complicated modifications discussed at the outset in order to integrate the transgene itself into the chromosomal DNA are inapplicable. For instance, a once-produced microorganism strain, which is prepared for a defined inactivation of the translocation apparatus, can be used for ever new transformations using similarly constructed vectors, which each time make available the same function curing the gene defect, but in each case carry various transgenes. A selection system which is very practical and can be employed in a versatile manner is thus available.

It is preferred that the genetic element used in the selection process of the invention be stable in the cell over a number of generations. Most preferably, this element contains a transgene and encodes a protein capable of compensating (i.e., curing) the translocation activity which is inactivated in a). This, then, is the technically most important field of application of selection systems. The genetic element carrying the transgene is stable over a number of generations, in particular one whose gene product is of commercial interest. Preferred embodiments thereof are carried out further below.

In preferred embodiments, a selection process according to the invention comprises the use of nucleic acids encoding proteins responsible for the essential translocation activity of one the following factors: SecA, SecY, SecE, SecD, SecF, signal peptidase, b-SRP (Ffh or Ffs/Scr), FtsY/Srb, PrsA or YajC.

As compiled in Table 1, these essential factors or the associated genes are those previously identified in E. coli or from B. subtilis. It is therefore straightforward, in particular in these two organisms, but also in related or even less related species, to establish a selection system according to the invention by identifying homologs encoding these factors. Since it is known that individual members of these genes can substitute the function concerned in other organisms, that is to say over and beyond the limit gram-negative/gram-positive, at least individual members of the genes concerned even from only distantly related species should be employable according to the invention.

Preferably, the essential translocation activity is one associated with one of the following subunits of the preprotein translocase: SecA, SecY, SecE, SecD or SecF, preferably the subunit SecA.

These factors then to a certain extent represent, as shown in FIG. 1, the functional core of the translocation apparatus. For SecA, it has been explained further above that this factor occupies an important key position in the ATPase activity. Thus, the selection process of the invention has been exemplified using the gene encoding SecA.

Preferably, selection processes according to the invention are characterized in that the curing according to (b) takes place by means of an activity acting identically to the inactivated endogenously present essential translocation activity, preferably by means of a genetically related activity, particularly preferably by means of the same activity.

It is reflected therein that on account of the generally high homology values between the species for the factors concerned, the genes from less closely related species concerned can also be employed. However, of course those from more closely related species and very particularly from the same organisms are preferred, because these are the most promising with respect to the crossing-over necessary for inactivation. It may again be pointed out that only a single gene suitable for the inactivation suffices in order to achieve a selection according to the invention.

As mentioned above, the DNA and amino acid sequences concerned are obtainable from generally accessible data banks. For instance, the sequences for the protein SecA from B. subtilis from the data bank “Subtilist” of the Pasteur Institute (see above) indicated in the sequence listing under SEQ ID NO. 1 and 2 have been retrieved (date: 2. 3. 2003); they are identical with that of Swiss-Prot (see above) which are deposited there under the accession number P28366.

The sequences indicated in the sequence protocol under SEQ ID NO. 3 and 4 for the protein SecA from E. coli originate from the data bank “Colibri” of the Pasteur Institute (see above; date: 2.3.2003); they are identical to that of Swiss-Prot (see above), which can be retrieved there under the accession number P10408.

SEQ ID NO. 5 and 6 for B. licheniformis were obtained from the commercially obtainable strain B. licheniformis (DSM13) as described in example 1 of the present application (Deutsche Sammlung von Mikroorganismen und Zellkulturen GmbH, Mascheroder Weg lb, 38124 Brunswick; www.dsmz.de).

Inasmuch as the species Bacillus subtilis, Escherichia coli and Bacillus licheniformis are most often employed in industrial applications, it is particularly important to make available a process according to the invention for these bacteria. Using the sequence listing for the present application, the homologous SecA genes from these three most important organisms are made available without them having to be isolated for copying. By means of these genes, it is, for example, possible to identify the homologs concerned in other microorganisms, for example by means of preparation of a gene bank and screening using one of these genes as a probe. In particular in related species, it is, however, also possible to employ these genes themselves for an inactivation according to the invention.

Preferred embodiments are thus characterized in that the curing according to (b) takes place by means of the regions of the gene SecA from Bacillus subtilis, Escherichia coli and Bacillus licheniformis restoring the translocation activity, which are indicated in the sequence listing under SEQ ID NO. 1, SEQ ID NO. 3 and SEQ ID NO. 5 respectively.

Preferred processes are moreover characterized in that the inactivation according to (a) takes place such that a recombination between the gene region inactivated according to (a) and the homologous region on the vector according to (b) is prevented or is not possible. It is preferred that the sequence encoding the essential translocation activity be completely deleted from the chromosomal gene concerned.

If the vector was integrated into the chromosome of the host cell, the lethal mutation would be permanently cured without a selection pressure on the vector concerned existing simultaneously. By this means, the actually interesting transgene could be lost by means of the following cell divisions. Extensive deletion during the inactivation step (a) prevents this.

In the prior art, in particular in the publication “Genetic manipulation of Bacillus amyloliquefaciens” by J. Vehmaanpera et al. (1991) in J. Biotechnol., Volume 19, pages 221-240, processes for the inactivation of genes by means of a deletion vector are described. With the aid of this description, it was possible in example 3 to carry out the deletion of the gene SecA from B. licheniformis successfully. The replication origin of this deletion vector is distinguished by its temperature dependence. It is particularly easily possible thereby first to select on a successful transformation at relatively low temperature and subsequently, by increasing the temperature, to exert a selection pressure on a successful integration, that is to say inactivation of the endogenous gene. Analogously, for example, a construct regulated by means of the addition of low molecular weight compounds would also be possible.

Preferred processes according to the invention are consequently characterized in that the inactivation according to (a) is carried out by means of a deletion vector, preferably by means of a deletion vector having an externally regulatable replication origin, particularly preferably by means of a deletion vector having a temperature-dependent replication origin.

As explained above, in principle it is possible that the curing vector according to (b), including the transgene, is integrated into the bacterial chromosome. Using this approach, concerns exist relating to loss of the transgene. Thus, preferred processes are characterized in that the vector according to (b) is a plasmid autonomously replicating in the microorganism which establishes itself in the derived cell line.

It is particularly advantageous if the plasmid is a plasmid which establishes itself in plural copy number (for example 2 to 100 plasmids per cell), preferably in a multiple copy number (more than 100 plasmids per cell). Increased numbers of plasmid copies enhances the curing step. Moreover, this approach increases production of the protein encoded by the transgene of interest, when present, thereby increasing the yield of protein via a gene dose effect.

Due to the great importance of gram-negative strains of bacteria, in particular in the cloning and characterization of genes or gene products, preferred selection processes are characterized in that the microorganism is a gram-negative strain of bacteria.

Among these, in particular, are to be understood processes which are include the use of a gram-negative strain of bacteria of the genera E. coli or Klebsiella, in particular derivatives of Escherichia coli K12, of Escherichia coli B or Klebsiella planticola, and very particularly derivatives of the strains Escherichia coli BL21 (DE3), E. coli RV308, E. coli DH5α, E. coli JM109, E. coli XL-1 or Klebsiella planticola (Rf). These are the organisms most frequently employed in molecular biology.

Gram-positive bacteria are of particular importance for fermentative protein production, particularly for production of secreted proteins. Preferred processes according to the invention are therefore characterized in that the microorganism is a gram-positive strain of bacteria.

Among these, in particular in industry, gram-positive strains of bacteria of the genera Staphylococcus, Corynebacteria or Bacillus are established, in particular of the species Staphylococcus carnosus, Corynebacterium glutamicum, Bacillus subtilis, B. licheniformis, B. amyloliquefaciens, B. globigii or B. lentus, and very particularly derivatives of the strains B. licheniformis or B. amyloliquefaciens, which is why these characterize correspondingly preferred selection processes.

Processes directed at high level production of proteins of commercial interest in microorganisms are of particular interest. Correspondingly preferred selection processes are thus those which are characterized in that the transgene according to (b) is one which codes for a nonenzyme protein, in particular for a pharmacologically relevant protein, very particularly for insulin or calcitonin.

However, enzymes are also of great industrial importance. Thus, according to the invention those processes are also encompassed which are characterized in that the transgene according to (b) is one which codes for an enzyme, preferably for a hydrolytic enzyme or an oxidoreductase, particularly preferably for a protease, amylase, hemicellulase, cellulase, lipase, cutinase, oxidase, peroxidase or laccase.

As mentioned previously, large-scale fermentation for production of the protein of interest is preferred. Also mentioned are the disadvantages of antibiotic selection (e.g., expense and environmental concerns), and auxotrophy based selection due to the ready compensation of metabolic defects due the nutrient complexity of industrial media.

The conversion of a selection process according to the invention to a large-scale process is therefore of particular importance, for example for the production of low molecular weight compounds such as antibiotics or vitamins or very particularly for protein production.

Processes for the production of a protein by culturing cells of a microorganism strain are generally known in the prior art. Production of the protein of interest naturally or after transformation with the gene encoding the protein of interest are cultured in a suitable manner and, where appropriate, stimulated for the formation of the protein of interest.

Thus, in accordance with another aspect of the invention, processes for the production of a protein by culturing cells of a microorganism selected via the methods described herein are disclosed. In preferred embodiments, the curing vector of b) contains a transgene and this preferably codes for a non-enzyme protein or for an enzyme. Among these, commercially important proteins are particularly preferred. Thus, proteins of interest include, without limitation, transgenically produced insulin, for the treatment of diabetes, and a broad spectrum of enzymes, e.g., proteases, lipases and amylases including, without limitation, oxidative enzymes employed for the production of detergents and cleansers.

In principle, bacteria can be used on a solid surface. This is in particular of importance for testing their metabolic properties or for permanent culture on the laboratory scale. For the production of proteins, on the other hand, processes are preferred which are characterized in that the culture of the microorganisms takes place in a liquid medium, preferably in a fermenter. Techniques of this type are facilitated by the selection methods based on the inactivation of essential translocation factors as disclosed herein.

Of particular importance are protein production processes wherein the protein of interest is secreted into the surrounding medium. This approach facilitates the workup of the product. A possible alternative according to the invention, however, also consists in breaking down the cells concerned producing the protein following the actual production and thereby obtaining the product.

In principle, any molecular biological alteration gives rise to a new strain of microorganism. Thus, new microorganism strains produced by the transformation and selection methods described herein are within the scope of the invention. In one embodiment, those new strains which differ from the starting strain (to put it more precisely: from the starting cell) by the specific inactivation of an essential translocation activity and its curing by provision of an identically acting translocation factor are provided. Novel microorganisms are thus produced by use of a selection process according to invention.

A particularly advantageous aspect consists in the fact that a group-related microorganism is obtained by always carrying out the same type of inactivation and curing on the curing vector but each time preparing another transgene. A process, once used successfully, can in this way be transferred to innumerable other selection problems.

For the realization, in particular, of the protein production processes explained above, it is necessary that the transgene is expressed. In preferred processes, the protein is secreted.

As mentioned previously, the selection methods of the invention are based on the essential nature of genes encoding the translocation apparatus. Use of genes of this type has not been considered as a means to select recombinant organisms, although numerous of these are known from a large number of microorganisms. Precisely this knowledge works to the advantage of selection systems according to the invention, since virtually all microorganisms possess such genes and can thus be identified using the selection methods described. For this, such genes have only to be inactivated as explained above and substituted in the cell concerned by a functioning homolog.

One aspect of the invention entails the use of a gene coding for an essential translocation activity for the selection of a microorganism. An exemplary use of such a gene comprises,
(a) inactivation of an endogenous, essential translocation activity in a target microorganism, and
(b) curing the inactivation of the essential translocation activity via transformation with a vector which contains a nucleic acid encoding a protein having said essential translocation activity.
   Most preferably, the vector used for curing in b) contains a transgene.

Preferably, the essential translocation activity is provided by a nucleic acid encoding one of the following factors: SecA, SecY, SecE, SecD, SecF, signal peptidase, b-SRP (Ffh or Ffs/Scr), FtsY/Srb, PrsA or YajC.

Among these, any use is preferred which is based on the essential translocation activity of one of the following subunits of the preprotein translocase: SecA, SecY, SecE, SecD or SecF, preferably the subunit SecA.

Preferably, the curing according to (b) is effected by providing an activity acting identically to the inactivated endogenously present essential translocation activity, preferably by means of a genetically related activity, particularly preferably via the same activity.

The present application exemplifies the use of the regions of the gene SecA from Bacillus subtilis, Escherichia coli or Bacillus licheniformis restoring the translocation activity for the curing according to step (b) of the present method. Sequences appropriate for this method include SEQ ID NO. 1, SEQ ID NO. 3 and SEQ ID NO. 5 respectively.

In particularly preferred embodiments, the vector according to (b) is a plasmid autonomously replicating in the microorganism. More preferably, the plasmid is established in the target microorganism in a plural, preferably in a multiple, copy number.

Finally the present invention is also realized by the provision of appropriate vectors. Vectors are intended hereby which carry a gene for an essential translocation activity and a transgene capable of expression which, however, when present as a single transgene, does not code for an antibiotic resistance.

The prior art describes, in connection with the characterization of the translocation proteins which can be used according to the invention vectors encoding the transloction protein which also contain antibiotic resistance markers. Such protein translocation molecules have been sequenced and cloned, namely by means of the common cloning vectors in the prior art which are known to contain markers encoding for antibiotic resistance. Thus, vectors comprising genes for an essential translocating enzyme and an antibiotic marker are known in the prior art. However, the use of such vectors for selection of microorganisms capable of producing a transgene has not been described.

Accordingly, a vector according to the invention is one in which the transgene contained is intended for protein production, codes for a pharmacologically relevant nonenzyme protein or for a hydrolytic enzyme or for an oxidoreductase. Such coding sequences require the presence of a functioning promoter. Here, of course, all such constructs are included in the scope of protection which also code for—possibly pharmacologically interesting—factors, which can mediate antibiotic resistance provided the presence of this vector is selected not by means of this property but by means of the essential translocation activity.

According to the details of the selection system, certain vectors also represent preferred embodiments of the present invention.

These include vectors encoding proteins which are able to cure the inactivated, endogenous, essential translocation in a microorganism strain, preferably by means of a genetically related activity, particularly preferably by means of the same activity.

Most preferably, these vectors include nucleic acids encoding the the essential translocation activity of one of the following factors: SecA, SecY, SecE, SecD, SecF, signal peptidase, b-SRP (Ffh or Ffs/Scr), FtsY/Srb, PrsA or YajC.

A more preferred embodiment comprises vectors which provide the essential translocation activity of one or more of the following subunits of the pre-protein translocase: SecA, SecY, SecE, SecD or SecF, preferably the subunit SecA.

According to the teaching of the present application, the vectors are furthermore preferred which are characterized in that the essential translocation activity is a SecA gene from Bacillus subtilis, Escherichia coli or Bacillus licheniformis, which are indicated in the sequence listing under SEQ ID NO. 1, SEQ ID NO. 3 and SEQ ID NO. 5 respectively.

It is preferred that the vectors are plasmids replicating autonomously in the microorganism used.

In this connection, it is particularly advantageous if the plasmids are plasmids capable of establishing a plural, preferably in a multiple, copy number.

### EXAMPLES

All molecular biological operations follow standard methods, such as are indicated, for example, in the handbook by Fritsch, Sambrook and Maniatis “Molecular cloning: a laboratory manual”, Cold Spring Harbor Laboratory Press, New York, 1989, or comparable relevant works. Enzymes and kits were employed according to the details of the respective manufacturer.

### Example 1

Isolation of the Gene secA from B. licheniformis

Identification of the secA Locus in B. licheniformis

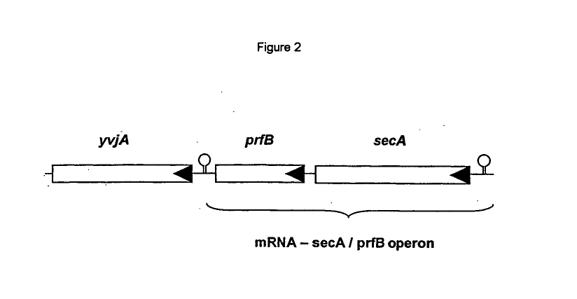

For the identification of the secA/prfB locus in B. licheniformis, a gene probe was derived by means of PCR with the aid of the known sequence of the prfB-secA gene locus of B. subtilis (databank “Subtilist” of the Pasteur Institute, 25, 28 rue du Docteur Roux, 75724 Paris CEDEX 15, France; genolist.pasteur.fr/SubtiList/; date: 8.16.2002). This gene locus is also shown in FIG. 2. The probe obtained was 3113 bp long and additionally comprised the first 451 bp of the N-terminal region of the gene prfB. Subsequently, preparations of chromosomal DNA of B. licheniformis, which is obtainable, for example, from Deutsche Sammlung von Mikroorganismen und Zellkulturen GmbH, Mascheroder Weg lb, 38124 Brunswick (www.dsmz.de) under the order number 13, and, for the control, chromosomal DNA of B. subtilis were digested using various restriction enzymes and subjected to a Southern hybridization using the probe mentioned. On the chromosomal DNA of B. licheniformis treated with the restriction enzyme MunI, a single fragment of a size of about 5.5 kB was identified, while the digestion of the chromosomal DNA of B. subtilis using MunI yielded the fragments expected for B. subtilis.

Cloning of the Identified Region from B. licheniformis DSM 13

Chromosomal DNA of the same strain B. licheniformis was isolated, digested preparatively using MunI and the DNA region around 5.5 kB was isolated by means of agarose gel electrophoresis, and the nucleic acids were extracted therefrom using commercially obtainable kits. The mixture of MunI-cleaved DNA fragments obtained was ligated in the MunI-compatible EcoRI cleavage site of the low-copy-number vector pHSG575 (described in: “High-copy-number and low-copy-number vectors for lacZ alpha-complementation and chloramphenicol- or kanamycin-resistance selection”; S. Takeshita; M. Sato; M. Toba; W. Masahashi; T. Hashimoto-Gotoh; Gene (1987), Volume 61, pages 63-74) and transformed in E. coli JM109 (obtainable from Promega, Mannheim, Germany).

Selection of the resistance encoded by the vector was carried out. Additionally, the method of blue/white screening (selection plates contained 80 μg/ml of X-Gal) served for the identification of clones which had taken up a vector having an insert. Moreover, 200 clones were obtained, of which it was possible by means of colony hybridization to identify 5 clones which carried the B. licheniformis-SecA gene. These were checked by subsequent Southern blot analysis using the probe described above and a vector derived from pHSG575 containing the MunI fragment carrying the SecA gene of B. licheniformis and comprising 5.5 kB was carried on under the name pHMH1.

Restriction Analysis

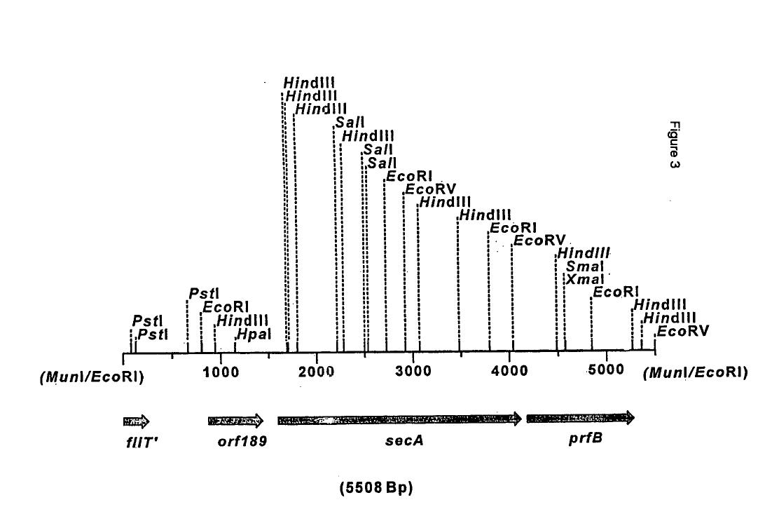

The cloned 5.5 kB region was first characterized by means of restriction mapping. For this, using various enzymes, individual and double digestions of pHMH1 were carried out and by means of Southern blot analysis those fragments were identified which carry parts of the SecA/prfB operon. The restriction map resulting therefrom was supplemented after complete sequencing of the 5.5 kB fragment (see below) and is shown in FIG. 3.

Sequence Analysis

The 5.5 kB fragment (FIG. 3) was sequenced into subsequences according to standard methods. The subsequences showed strong homologies with the following genes from B. subtilis: fliT (encoding a flagellar protein), orf189/yvyD (unknown function), SecA (translocase-binding subunit; ATPase) and prfB (peptide chain release factor 2), in exactly the same gene sequence as in B. subtilis. These are likewise shown in FIG. 3.

On the basis of the foregoing, it appears that SecA from B. licheniformis exerts the same biochemical activity as, in particular, the SecA from B. subtilis and thereby provides the same physiological function. It is thus to be considered as an essential enzyme in the translocation process.

The DNA sequence and the amino acid sequence determined according to this example are given in the sequence listing as SEQ ID NO. 5 or 6 respectively. Accordingly, the translation start lies in the position 154 and the stop codon in the positions 2677 to 2679. The subsequence from the positions 60 to 65 or 77 to 82 is presumably to be regarded as a promoter region and the region from position 138 to 144 as a ribosome binding site.

### Example 2

Preparation of a Plasmid Containing a SecA Gene and Subtilisin Gene

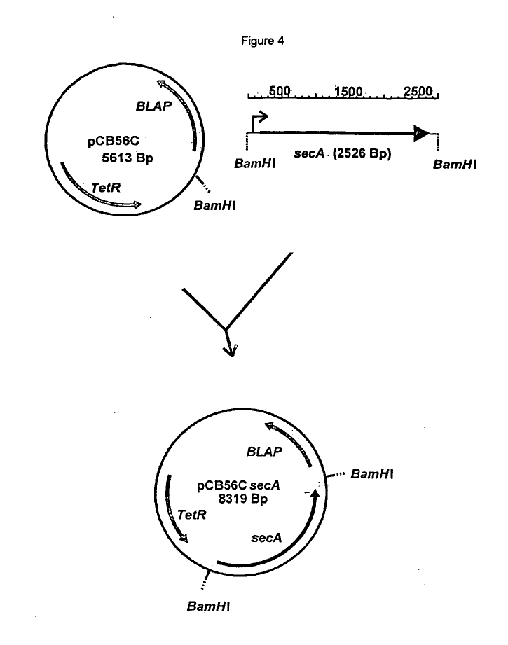

The SecA gene obtained according to Example 1 was amplified using its own promoter by means of PCR starting from chromosomal DNA from B. licheniformis. For this, as shown in FIG. 4, with the aid of the DNA sequence of the gene of B. licheniformis, primers were selected which at the respective 5′-end possess a BamHI restriction cleavage site. By means of this, the fragment amplified using these primers was cloned into the cleavage site of the plasmid pCB56C. This is described in the application WO 91/02792 A1 and contains the gene for the alkaline protease from B. lentus (BLAP).

This cloning strategy, also shown in FIG. 4, yielded the vector pCB56CSecA 8319 bp in size which, in addition to the genes SecA and BLAP, also contains one which codes for a tetracycline resistance.

This vector pCB56CSecA and, for the control, the starting vector pCB56C were transformed in B. licheniformis, mainly in the case of pCB56C in the wild-type strain B. licheniformis (SecA) capable of the formation of SecA. In the case of pCB56CSecA, the transformation was carried out such that the endogenous SecA was simultaneously inactivated. The procedure for this is described in Example 3.

The two strains B. licheniformis (ΔSecA) pCB56CSecA and B. licheniformis (SecA) pCB56C were obtained as described above, which were both able to express the plasmid-encoded gene for the alkaline protease. They are further characterized as described in Example 4.

### Example 3

Preparation of the Strain B. licheniformis (ΔSecA) pCB56CSecA

The switching off of the gene SecA was performed by means of a deletion vector. The procedure follows the description of J. Vehmaanperä et al. (1991) in J. Biotechnol., Volume 19, pages 221-240.

The vector selected for SecA deletion was the plasmid pE194 described in the same publication. The advantage of this deletion vector is that it possesses a temperature-dependent replication origin. At 33° C., pE194 can replicate in the cell, such that a successful transformation can first be selected at this temperature. Subsequently, the cells which contain the vector are incubated at 42° C. At this temperature, the deletion vector no longer replicates and a selection pressure is exerted on the integration of the plasmid into the chromosome by means of one of the two homologous regions (up- or downstream region of SecA). A further homologous recombination by means of the other (second) homologous region then leads to the deletion of SecA. A repeated recombination of the first homologous region would also be possible. In this connection, the vector recombines again from the chromosome, such that the chromosomal SecA is retained. The SecA deletion must therefore be detected in the Southern blot after restriction of the chromosomal DNA using suitable enzymes or with the aid of the PCR technique by means of the size of the amplified region.

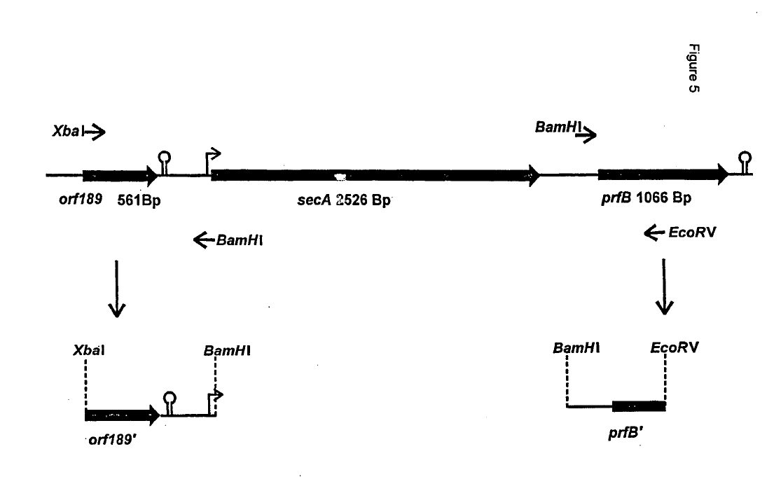

For the construction of the deletion vector, the regions located up- and downstream of SecA (FIG. 5) were amplified by means of PCR. The primers for the amplification and the restriction cleavage sites for subsequent cloning (XbaI and EcoRV) associated with these were selected with the aid of the DNA sequence of the SecA/prfB locus of B. licheniformis determined according to Example 1. In the case of the SecA deletion, it should be taken into consideration that the prfB located downstream of SecA lies in one operon with SecA, that is possesses no promoter of its own (compare FIG. 2). The prfB codes for the protein RF2, which in connection with the protein biosynthesis ensures the detachment of the protein from the ribosome. In order to guarantee the transcription of the prfB, which is important for protein biosynthesis, even after SecA deletion, the orf189 with its own terminator situated before the SecA and the SecA promoter located downstream was amplified such that the prfB can be transcribed directly from the SecA promoter after SecA deletion (FIG. 5).

The amplified regions (orf189′ and prfB′) were intercloned into the E. coli vector pBBRMCS2 in a control step. The subsequent sequencing of the orf189′ prfB′ construct showed that the amplified fragments were cloned together correctly.

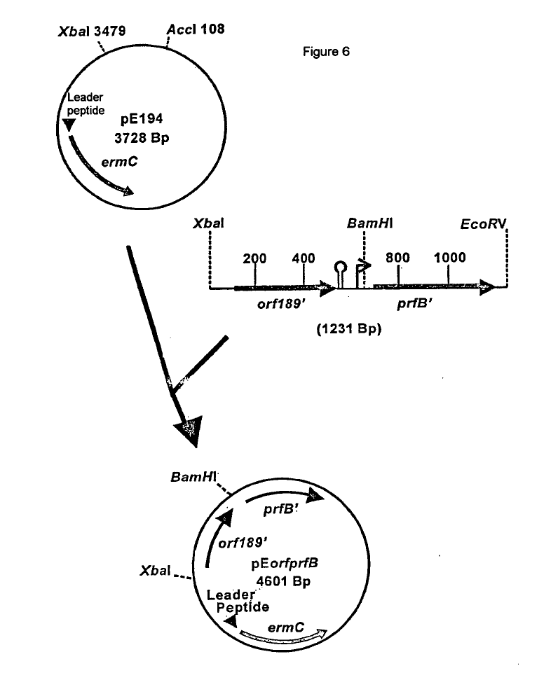

The orf189‘prfB’ construct was recloned in the next step into the vector pE194 in B. subtilis DB104 selected for the deletion (FIG. 6). In this context, using the method of protoplast transformation according to Chang & Cohen, 1979, transformants were obtained which carried the deletion vector pEorfprfB. All operations were carried out at 33° C. in order to guarantee replication of the vector.

In a next step, the vector pCB56CSecA described in Example 2 was likewise transformed into the host strain B. licheniformis carrying the plasmid pEorfprfB by means of the method of protoplast transformation. The transformants obtained in such a way and identified as positive using customary methods were subsequently selected for the presence of both plasmids at 42° C. under selection pressure (tetracycline for pCB56CSecA and erythromycin for pEorfprfB). At this temperature, the deletion vector can no longer replicate and only those cells in which the vector is integrated into the chromosome survive, this integration taking place with the highest probability in homologous or identical regions. By culturing at 33° C. without erythromycin selection pressure, the excision of the deletion vector can subsequently be induced, the chromosomally encoded gene SecA being removed from the chromosome completely.

The plasmid pCB56CSecA, which mediates the ability for subtilisin synthesis and also makes available the essential translocatlon factor SecA, remains in the cell. The strain obtained in this manner was designated by B. licheniformis (ΔSecA) pCB56CSecA.

### Example 4

Investigation of the Plasmid Stability

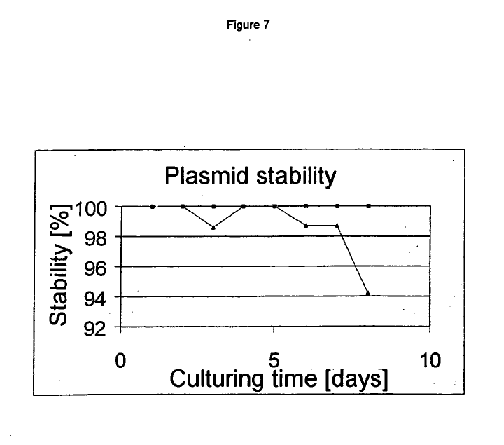

For the determination of the genetic stability of the SecA-carrying subtilisin plasmid pCB56CSecA, the two strains B. licheniformis (SecA) pCB56C and B. licheniformis (ΔSecA) pCB56CSecA obtained according to Examples 2 and 3 were investigated in liquid medium in a shaker flask experiment without addition of antibiotic. For this, starting from one individual colony each, an overnight culture was grown and using 14 ml of LB medium in each case (according to standard recipe) were inoculated to an optical density at 600 nm (OD₆₀₀) of 0.05. Culturing was carried out in a 100 ml Erlenmeyer shaker flask. After 8 to 16 hours in each case, the cultures were inoculated into 14 ml of fresh medium and here in turn an OD₆₀₀ of 0.05 was set. The culturing was carried out over 8 days and nights; the cultures were inoculated altogether 16 times in this process. Every day, dilution series were plated out and a random selection of the clones obtained was transferred to protease test plates. The result is shown in Table 2 and in FIG. 7.

TABLE 2
Plasmid stability in the transformants B. licheniformis (SecA) pCB56C (control) and
B. licheniformis (ΔSecA) pCB56CSecA, detectable with the aid of the respective
fraction of the clones with protease activity

| | B. licheniformis (SecA) | | | B. licheniformis (ΔSecA) | | |
|---|---|---|---|---|---|---|
| | pCB56C | | | pCB56CSecA | | |
| | Number of | | Fraction of the | Number of | | Fraction of the |
| | tested clones | | clones with | tested clones | | clones with |
| Time | | without | protease activity | | without | protease activity |
| [days] | total | protease activity | [%] | total | protease activity | [%] |
| 1 | 72 | 0 | 100 | 72 | 0 | 100 |
| 2 | 72 | 0 | 100 | 70 | 0 | 100 |
| 3 | 72 | 1 | 98.6 | 49 | 0 | 100 |
| 4 | 52 | 0 | 100 | 52 | 0 | 100 |
| 5 | 78 | 0 | 100 | 78 | 0 | 100 |
| 6 | 78 | 1 | 98.7 | 78 | 0 | 100 |
| 7 | 78 | 1 | 98.7 | 77 | 0 | 100 |
| 8 | 104 | 6 | 94.2 | 104 | 0 | 100 |

For each culturing time, all clones of the strain B. licheniformis (ΔSecA) pCB56CSecA show protease activity, while with the strain B. licheniformis (SecA) pCB56C individual clones no longer possess any protease activity. This is to be interpreted as a loss of the plasmid pCB56C; this loss was additionally checked by plasmid minipreparation.

These data clearly show that on culturing in LB medium without antibiotic addition, in particular without tetracycline, for which the plasmid would impart resistance, the SecA-carrying subtilisin plasmid pCB56CSecA is stable in the ΔSecA strain, while the subtilisin plasmid pCB56C in the strain without chromosomal SecA deletion is lost in the course of culturing. The gene SecA from B. licheniformis can thus cure the chromosomal SecA deficiency on transfer to an expression vector and in this manner can be utilized for the selection of a bacterial culture which expresses a gene for another protein, in this case an alkaline protease.

1. A process for the selection of a microorganism, comprising:
(a) inactivating an endogenous gene which encodes an essential translocation activity in said microorganism; and
(b) introducing a vector into said microorganism which encodes a protein comprising said essential translocation activity, thereby curing the inactivation of said translocation activity, said vector optionally further comprising a transgene.

2. The process as claimed in claim 1, wherein the vector of b) contains a transgene, said trangene encoding a protein.

3. The process as claimed in claim 1, wherein said essential translocation activity is expressed from a nucleic acid which encodes a factor selected from the group consisting of SecA, SecY, SecE, SecD, SecF, signal peptidase, b-SRP (Ffh or Ffs/Scr), FtsY/Srb, PrsA or YajC.

4. The process as claimed in claim 4, wherein said essential translocation activity is encoded by at least one subunit of the preprotein translocase selected from the group consisting of SecA, SecY, SecE, SecD or SecF.

5. The process of claim 4, wherein said subunit is SecA.

6. The process as claimed in claim 1, wherein said vector of b) comprises a nucleic acid which encodes a protein having the essential translocation activity inactivated in step a) or a nucleic acid encoding a homolog thereof.

7. The process as claimed claim 1 wherein the curing according to (b) is effected by introducing nucleic acid encoding SecA, said nucleic acid being selected from the group consisting of SEQ ID NO: 1, SEQ ID NO: 3 and SEQ ID NO: 5.

8. The process as claimed in claim 1, wherein the inactivation according a) results in a deletion of the endogenous nucleic acid sequence encoding the essential translocation activity, such that recombination between the curing vector of b) and the homologous chromosomal region inactivated is prevented.

9. The process as claimed in claim 8, wherein said prevention results from a complete loss of the nucleic acid encoding said essential translocation from the chromosome of the microorganism to be selected.

10. The process as claimed in 1, wherein the inactivation according to (a) is effected by a deletion vector which causes deletion of an endogenous nucleic acid encoding a protein having said essential translocation activity.

11. The process as claimed in claim 10, wherein said deletion vector comprises an externally regulatable replication origin.

12. The process as claimed in claim 11 wherein said externally regulatable replication origin is temperature-sensitive.

13. The process as claimed in claim 1, wherein the vector according to (b) is a plasmid which replicates autonomously in the microorganism.

14. The process as claimed in claim 13, wherein said plasmid is a multiple copy number plasmid.

15. The process as claimed in claim 1, wherein said microorganism is a gram-negative strain of bacteria.

16. The process as claimed in claim 15, wherein said gram-negative strain of bacteria is selected from the group consisting of E. coli, Klebsiella, Escherichia coli K12, Escherichia coli B, Klebsiella planticola, Escherichia coli BL21 (DE3), E. coli RV308, E. coli DH5α, E. coli JM109, E. coli XL-1 and Klebsiella planticola (Rf).

17. The process as claimed in claim 1, wherein said microorganism is a gram-positive strain of bacteria.

18. The process as claimed in claim 17, wherein said gram-positive strain of bacteria is selected from the group consisting of Staphylococcus, Corynebacteria, Bacillus, Staphylococcus carnosus, Corynebacterium glutamicum, Bacillus subtilis, B. licheniformis, B. amyloliquefaciens, B. globigii, B. lentus, or derivatives thereof.

19. The process as claimed in claim 2, wherein said transgene encodes an enzyme selected from the group consisting of a hydrolytic enzyme, an oxidoreductase, a protease, amylase, hemicellulase, cellulase, lipase, cutinase, oxidase, peroxidase, and a laccase.

20. The process as claimed in claim 2, wherein said transgene encodes a pharmacologically relevant protein lacking enzymatic activy.

21. The processs as claimed in claim 20, wherein said protein is selected from the group consisting of insulin and calcitonin.

22. A process for the preparation and isolation of a protein of interest comprising selecting the microorganism for production by
(a) inactivating an endogenous gene which encodes an essential translocation activity in said microorganism;
(b) introducing a vector into said microorganism which encodes a protein comprising said essential translocation activity, thereby curing the inactivation of said translocation activity, said vector comprising a transgene encoding said protein protein of interest under conditions where said protein is produced; and
c) isolating said protein of interest.

23. The process as claimed in claim 22, wherein said microorganism is cultured in liquid medium, optionally in a fermenter.

24. The process as claimed in claim 22 wherein the protein of interest is secreted into the surrounding medium.

25. A microorganism, obtainable by the selection process as claimed in claim 1.

26. The microorganism as claimed in claim 25, characterized in that the transgene is expressed.

27. The microorganism as claimed in claim 25, characterized in that the transgene is secreted.

28. A vector for use in the process of claim 1, comprising a gene encoding a protein having an essential translocation activity and a transgene which, when present as the only transgene, does not code for antibiotic resistance.

29. The vector as claimed in claim 28, wherein said transgene encodes a protein selected from the group consisting of a pharmacologically relevant nonenzyme protein, a hydrolytic enzyme, and an oxidoreductase.

30. The vector as claimed in claim 28, wherein said protein having said essential translocation activity is identical to the protein inactivated in a) or a closely related homolog thereof

31. The vector as claimed in claim 28, wherein said essential translocation activity is encoded by a nucleic acid encoding at least one factor selected from the group consisting of SecA, SecY, SecE, SecD, SecF, signal peptidase, b-SRP (Ffh or Ffs/Scr), FtsY/Srb, PrsA or YajC.

32. The vector as claimed in claim 28, wherein said essential translocation activity is encoded by a nucleic acid encoding at least one subunit of the preprotein translocase selected from the group consisting of SecA, SecY, SecE, SecD and SecF.

33. The vector as claimed in claim 28, wherein said essential translocation activity is encoded by a nucleic acid encoding SecA selected from the group consisting of SEQ ID NO. 1, SEQ ID NO. 3 and SEQ ID NO. 5.

34. The vector as claimed in claim 28, wherein said vector is a plasmid replicating autonomously in the microorganism.

35. The vector as claimed in claim 30, wherein said plasmid is a multiple, copy number plasmid.

36. The process as claimed in claim 22, wherein said transgene encodes a protein selected from the group consisting of a hydrolytic enzyme, an oxidoreductase, a protease, amylase, hemicellulase, cellulase, lipase, cutinase, oxidase, peroxidase, a laccase, a pharmacologically relevant protein lacking enzymatic activy, insulin and calcitonin.


SEQUENCE LISTING (6 sequences)

SEQ ID NO: 1 - DNA, 4148 bp, Bacillus subtilis
  CDS (544)..(3069)
gtccgaggtgcataacgaggatatgtacaacgcaattgatctcgcaacaaacaaactggaacgtcaaatccgtaagcataaaacgaaagtaaaccgtaaattccgtgagcagggctctccaaaatatttattggcaaacggtcttggctctgatacagatattgcggttcaggatgacatagaagaggaggagagcttggacatcgtccgtcagaaacgctttaatttaaagccgatggatagtgaagaagcgatcttgcaaatgaatatgctcggccataatttctttgttttcacaaatgcggaaacaaaccttacaaatgtcgtgtaccgcagaaatgacgggaaatatggcttaattgaaccgactgaataatgaagagaagccttccgtgatgtccgcggaaggtttttgtttttcttatttgcaaattctttggaaataacaaaaggtatgatatgataatgagaggtatacatggactagtaaattatttatacatgcctctaaaataggcgtgtgatgatagaggagcgttataaatgcttggaattttaaataaaatgtttgatccaacaaaacgtacgctgaatagatacgaaaaaattgctaacgatattgatgcgattcgcggagactatgaaaatctctctgacgacgcattgaaacataaaacaattgaatttaaagagcgtcttgaaaaaggggcgacaacggatgatcttcttgttgaagctttcgctgttgttcgagaagcttcacgccgcgtaacaggcatgtttccgtttaaagtccagctcatggggggcgtggcgcttcatgacggaaatatagcggaaatgaaaacaggggaagggaaaacattaacgtctaccctgcctgtttatttaaatgcgttaaccggtaaaggcgtacacgtcgtgactgtcaacgaatacttggcaagccgtgacgctgagcaaatggggaaaattttcgagtttctcggtttgactgtcggtttgaatttaaactcaatgtcaaaagacgaaaaacgggaagcttatgccgctgatattacttactccacaaacaacgagcttggcttcgactatttgcgtgacaatatggttctttataaagagcagatggttcagcgcccgcttcattttgcggtaatagatgaagttgactctattttaattgatgaagcaagaacaccgcttatcatttctggacaagctgcaaaatccactaagctgtacgtacaggcaaatgcttttgtccgcacgttaaaagcggagaaggattacacgtacgatatcaaaacaaaagctgtacagcttactgaagaaggaatgacgaaggcggaaaaagcattcggcatcgataacctctttgatgtgaagcatgtcgcgctcaaccaccatatcaaccaggccttaaaagctcacgttgcgatgcaaaaggacgttgactatgtagtggaagacggacaggttgttattgttgattccttcacgggacgtctgatgaaaggccgccgctacagtgaggggcttcaccaagcgattgaagcaaaggaagggcttgagattcaaaacgaaagcatgaccttggcgacgattacgttccaaaactacttccgaatgtacgaaaaacttgccggtatgacgggtacagctaagacagaggaagaagaattccgcaacatctacaacatgcaggttgtcacgatccctaccaacaggcctgttgtccgtgatgaccgcccggatttaatttaccgcacgatggaaggaaagtttaaggcagttgcggaggatgtcgcacagcgttacatgacgggacagcctgttctagtcggtacggttgccgttgaaacatctgaattgatttctaagctgcttaaaaacaaaggaattccgcatcaagtgttaaatgccaaaaaccatgaacgtgaagcgcagatcattgaagaggccggccaaaaaggcgcagttacgattgcgactaacatggcggggcgcggaacggacattaagcttggcgaaggtgtaaaagagcttggcgggctcgctgtagtcggaacagaacgacatgaatcacgccggattgacaatcagcttcgaggtcgttccggacgtcagggagacccggggattactcaattttatctttctatggaagatgaattgatgcgcagattcggagctgagcggacaatggcgatgcttgaccgcttcggcatggacgactctactccaatccaaagcaaaatggtatctcgcgcggttgaatcgtctcaaaaacgcgtcgaaggcaataacttcgattcgcgtaaacagcttctgcaatatgatgatgttctccgccagcagcgtgaggtcatttataagcagcgctttgaagtcattgactctgaaaacctgcgtgaaatcgttgaaaatatgatcaagtcttctctcgaacgcgcaattgcagcctatacgccaagagaagagcttcctgaggagtggaagcttgacggtctagttgatcttatcaacacaacttatcttgatgaaggtgcacttgagaagagcgatatcttcggcaaagaaccggatgaaatgcttgagctcattatggatcgcatcatcacaaaatataatgagaaggaagagcaattcggcaaagagcaaatgcgcgaattcgaaaaagttatcgttcttcgtgccgttgattctaaatggatggatcatattgatgcgatggatcagctccgccaagggattcaccttcgtgcttacgcgcagacgaacccgcttcgtgagtatcaaatggaaggttttgcgatgtttgagcatatgattgaatcaattgaggacgaagtcgcaaaatttgtgatgaaagctgagattgaaaacaatctggagcgtgaagaggttgtacaaggtcaaacaacagctcatcagccgcaagaaggcgacgataacaaaaaagcaaagaaagcaccggttcgcaaagtggttgatatcggacgaaatgccccatgccactgcggaagcgggaaaaaatataaaaattgctgcggccgtactgaatagttcgccccggcaagtttactgaacgcggcgcctgcaggctgcgatcttttaatgaggtgaatgaaatgaattatcagaaattagagcagagctcgaaaatatggcttctcgtttagcggactttagggggtctctttgacctcgaatcaaaggaggcccgcattgctgagctagatgaacaaatggctgatccggaattctggaatgatcagcaaaaagctcaaacggttataaatgaagcaaacggtttaaaggattatgtcaattcgtataaaaaattgaatgaatcccacgaagaattacaaatgactcatgatcttttgaaagaagagccggacactgatctccagcttgagcttgaaaaagaactaaagtcattaacaaaagagttcaatgagtttgagcttcagcttcttctcagcgagccgtatgataaaaataacgcgattttagaactgcaccctggtgctggcggtacagagtcacaggactggggctctatgcttcttagaatgtatacaagatggggagagcgccgcggctttaaagtagagactctcgattaccttccaggtgacgaggcgggaatcaagtcagtgacattgctcatcaaaggacacaacgcttacgggtatctcaaagcagaaaaaggtgttcatcgtcttgtgcggatctcaccatttgattcatcaggccgccgccacacatctttcgtttcatgtgaagtcatgcctgaatttaacgatgaaattgatattgatattcgtacggaggatattaaagttgacacgtaccgtgcaagcggcgcgggcggacagcacgtcaatacgacggattcagccgttcggattactcacttgccgacgaacgtagttgtgacatgccaaacggagcgctcacaaattaaaaaccgtgaaagagccatgaaaatgctgaaggccaagctgtatcagcgcagaattgaagagcagcaggcagagctggatgaaattcgcggtgaacaaaaagaaatcggctggggcagccaaatccgttcttatgtattccatccgtattccatggtaaaagaccatcgggac

SEQ ID NO: 2 - protein, 841 aa, Bacillus subtilis
MLGILNKMFDPTKRTLNRYEKIANDIDAIRGDYENLSDDALKHKTIEFKERLEKGATTDDLLVEAFAVVREASRRVTGMFPFKVQLMGGVALHDGNIAEMKTGEGKTLTSTLPVYLNALTGKGVHVVTVNEYLASRDAEQMGKIFEFLGLTVGLNLNSMSKDEKREAYAADITYSTNNELGFDYLRDNMVLYKEQMVQRPLHFAVIDEVDSILIDEARTPLIISGQAAKSTKLYVQANAFVRTLKAEKDYTYDIKTKAVQLTEEGMTKAEKAFGIDNLFDVKHVALNHHINQALKAHVAMQKDVDYVVEDGQVVIVDSFTGRLMKGRRYSEGLHQAIEAKEGLEIQNESMTLATITFQNYFRMYEKLAGMTGTAKTEEEEFRNIYNMQVVTIPTNRPVVRDDRPDLIYRTMEGKFKAVAEDVAQRYMTGQPVLVGTVAVETSELISKLLKNKGIPHQVLNAKNHEREAQIIEEAGQKGAVTIATNMAGRGTDIKLGEGVKELGGLAVVGTERHESRRIDNQLRGRSGRQGDPGITQFYLSMEDELMRRFGAERTMAMLDRFGMDDSTPIQSKMVSRAVESSQKRVEGNNFDSRKQLLQYDDVLRQQREVIYKQRFEVIDSENLREIVENMIKSSLERAIAAYTPREELPEEWKLDGLVDLINTTYLDEGALEKSDIFGKEPDEMLELIMDRIITKYNEKEEQFGKEQMREFEKVIVLRAVDSKWMDHIDAMDQLRQGIHLRAYAQTNPLREYQMEGFAMFEHMIESIEDEVAKFVMKAEIENNLEREEVVQGQTTAHQPQEGDDNKKAKKAPVRKVVDIGRNAPCHCGSGKKYKNCCGRTE

SEQ ID NO: 3 - DNA, 2706 bp, Escherichia coli
  CDS (1)..(2706)
atgctaatcaaattattaactaaagttttcggtagtcgtaacgatcgcaccctgcgccggatgcgcaaagtggtcaacatcatcaatgccatggaaccggagatggaaaaactctccgacgaagaactgaaagggaaaaccgcagagtttcgtgcgcgtctggaaaaaggcgaagtgctggaaaatctgatcccggaagctttcgccgtggtgcgtgaggcaagtaagcgcgtctttggtatgcgtcacttcgacgttcagttactcggcggtatggttcttaacgaacgctgcatcgccgaaatgcgtaccggtgaaggtaaaaccctgaccgcaacgctgcctgcttacctgaacgcactaaccggtaaaggcgtacacgtagttaccgtcaacgactacctggcgcaacgtgacgccgaaaacaaccgtccgctgtttgaattccttggcctgactgtcggtatcaacctgccgggcatgccagcaccggcaaagcgtgaagcctacgctgctgacatcacttacggtacgaacaacgaatacggctttgactacctgcgcgacaacatggcattcagccctgaagaacgtgtacaacgtaaactgcactatgcgctggtggacgaagtggactccatcctcatcgatgaagcgcgtacaccgctgatcatttccggcccagcagaagacagctcggaaatgtataaacgcgtgaataaaattattccgcacctgatccgtcaggaaaaagaagactccgaaaccttccagggcgaaggccacttctcggtggatgaaaaatctcgccaggtgaacctgaccgaacgtggtctggttctgattgaagaactgctggttaaagaaggcatcatggatgaaggtgagtctctgtactctccggccaacatcatgctgatgcaccacgtaacggcggcgctgcgcgctcatgcgctgtttacccgcgacgtcgactacatcgttaaagatggtgaagttatcatcgttgacgaacacaccggtcgtaccatgcagggccgtcgctggtccgatggtctgcaccaggctgtggaagcgaaagaaggtgtgcagatccagaacgaaaaccagacgctggcttcgatcaccttccagaactacttccgtctgtatgaaaaactggcggggatgactggtactgctgataccgaagctttcgaattcagctccatctataagctggatactgtcgttgttccgaccaaccgtccaatgattcgtaaagatctgccggacctggtctacatgactgaagcggaaaaaattcaggcgatcattgaagatatcaaagaacgtactgcgaaaggccagccggtgctggtgggtacaatctccatcgaaaaatcggagctggtgtcaaatgaactgaccaaagccggtattaagcacaacgtcctgaacgccaaattccatgccaacgaagcggcgattgttgctcaggcaggttatccggctgcggtgactatcgcgaccaacatggcgggtcgtggtaccgatattgtgctcggtggtagctggcaggcagaagttgccgcgctggaaaatccgactgcagagcaaattgaaaaaattaaagccgactggcaggtacgtcacgatgcggtactggcagcaggtggcctgcatatcatcggtactgaacgtcacgaatcccgtcgtatcgataaccagctgcgcggtcgttctggtcgtcagggggatgctggttcttctcgtttctacctgtcgatggaagatgcgctgatgcgtatttttgcttccgaccgagtatccggcatgatgcgtaaactgggtatgaagccaggcgaagccattgagcacccgtgggtgaccaaagcgattgccaacgcccagcgtaaagttgaaagccgtaacttcgacattcgtaagcaactgctggaatatgatgacgtggctaacgatcagcgtcgcgccatttactcccagcgtaacgaactgctggatgtcagcgatgtgagcgaaaccatcaacagcattcgtgaagatgtgttcaaagcgaccattgatgcctacattccgccacagtcgctggaagaaatgtgggatattccggggctgcaggaacgtctgaagaacgatttcgacctcgatttgccaattgccgagtggctggataaagaaccagaactgcatgaagagacgctgcgtgagcgcattctggcgcagtccatcgaagtgtatcagcgtaaagaagaagtggttggtgctgagatgatgcgtcacttcgagaaaggcgtcatgctgcaaactctcgactctctgtggaaagagcacctggcagcgatggactatctgcgtcagggtatccacctgcgtggctatgcacagaaagatccgaagcaggaatacaaacgtgaatcgttctccatgtttgcagcgatgctggagtcgttgaaatatgaagttatcagtacgctgagcaaagttcaggtacgtatgcctgaagaggttgaggagctggaacaacagcgtcgtatggaagccgagcgtttagcgcaaatgcagcagcttagccatcaggatgacgactctgcagccgcagctgcactggcggcgcaaaccggtgaacgcaaagtaggacgtaacgatccttgcccgtgtggttctggtaaaaaatacaagcagtgccatggccgcctgcaataa

SEQ ID NO: 4 - protein, 901 aa, Escherichia coli
MLIKLLTKVFGSRNDRTLRRMRKVVNIINAMEPEMEKLSDEELKGKTAEFRARLEKGEVLENLIPEAFAVVREASKRVFGMRHFDVQLLGGMVLNERCIAEMRTGEGKTLTATLPAYLNALTGKGVHVVTVNDYLAQRDAENNRPLFEFLGLTVGINLPGMPAPAKREAYAADITYGTNNEYGFDYLRDNMAFSPEERVQRKLHYALVDEVDSILIDEARTPLIISGPAEDSSEMYKRVNKIIPHLIRQEKEDSETFQGEGHFSVDEKSRQVNLTERGLVLIEELLVKEGIMDEGESLYSPANIMLMHHVTAALRAHALFTRDVDYIVKDGEVIIVDEHTGRTMQGRRWSDGLHQAVEAKEGVQIQNENQTLASITFQNYFRLYEKLAGMTGTADTEAFEFSSIYKLDTVVVPTNRPMIRKDLPDLVYMTEAEKIQAIIEDIKERTAKGQPVLVGTISIEKSELVSNELTKAGIKHNVLNAKFHANEAAIVAQAGYPAAVTIATNMAGRGTDIVLGGSWQAEVAALENPTAEQIEKIKADWQVRHDAVLAAGGLHIIGTERHESRRIDNQLRGRSGRQGDAGSSRFYLSMEDALMRIFASDRVSGMMRKLGMKPGEAIEHPWVTKAIANAQRKVESRNFDIRKQLLEYDDVANDQRRAIYSQRNELLDVSDVSETINSIREDVFKATIDAYIPPQSLEEMWDIPGLQERLKNDFDLDLPIAEWLDKEPELHEETLRERILAQSIEVYQRKEEVVGAEMMRHFEKGVMLQTLDSLWKEHLAAMDYLRQGIHLRGYAQKDPKQEYKRESFSMFAAMLESLKYEVISTLSKVQVRMPEEVEELEQQRRMEAERLAQMQQLSHQDDDSAAAAALAAQTGERKVGRNDPCPCGSGKKYKQCHGRLQ

SEQ ID NO: 5 - DNA, 2706 bp, Bacillus licheniformis
  CDS (154)..(2679)
gatccccctcccggatcttccgcagaggggattttttccgttcccccgcggtaaattgtttggaaatgacaaaaggtatgatatgatattgcatatataaaaattactgtttactcatgcttaaacaaggaaattaaagaggagcgttattctatgcttggaattttaaataaagtgtttgatccgacaaaacgcacgctcagccgttatgaaaagaaagcgaacgagattgatgcgctcaaggcagatatagagaagctttcagacgaagctttgaagcaaaagacgatcgagttcaaagagcgccttgaaaaaggcgaaacggttgacgatcttttggttgaagcgtttgccgttgtcagggaagcttcccggcgcgtgacaggcatgtttccgtttaaggttcagctgatggggggcgtcgcccttcatgaagggaatatcgccgaaatgaaaacgggggaaggtaaaacgctgacttccacaatgcccgtttacttgaacgctctgtcagggaaaggcgttcacgtcgtgacggtcaacgaatacctggcgagccgcgacgctgaagagatggggaaaatctttgagtttctcgggctgacggtcggcctaaacctgaacagcctgtcaaaagacgagaagcgtgaagcctatgcagcagatattacgtattctacgaataatgagcttggctttgactacttgcgcgacaacatggtgctttataaagagcagatggttcagcgcccgcttcattttgcggtcatcgatgaagtcgactccattttgatcgatgaagcaagaacgccgctcatcatttctggacaagcggccaaatccaccaagctttatgttcaggccaatgcgtttgtccgcacgctaaaagcggatcaggactacacatacgatgtgaaaacaaaaggcgttcagctgactgaagaggggatgacaaaagctgaaaaggcatttggcatcgaaaacttgtttgacgtccgccatgtcgccttaaaccatcatattgcccaggcgctgaaagcccatgcggcgatgcataaagacgtcgactacgtcgtcgaagacggtcaggtcgttatcgtcgactcttttacaggccgtttgatgaaaggccgccgctacagcgacggacttcaccaggccattgaagcgaaggaaggccttgagatccaaaatgagagcatgacgctcgcgacgatcaccttccagaactatttccgaatgtatgaaaaattggctggaatgacgggtaccgcaaaaacggaagaagaagaattccgcaacatctacaacatgcaggttgttacgattccgaccaacaagccgattgcccgcgatgaccgaccggatttaatttaccggaccatggaaggaaaatttaaagctgttgcagaggatgtcgcccagcgctatatggtcggacagccggtacttgtcggtacggttgcggttgaaacatctgaattgatatcaaggctccttaaaaataaaggaatcccgcatcaagtgttgaacgcgaaaaaccatgagcgggaagctcagattatcgaagatgccgggcaaaaaggcgcggtcaccatcgcgaccaacatggcgggccgcggaacggacatcaagcttggcgaaggtgtaaaagagcttggcggactggccgtcatcggtacggaacgccatgaatcaaggcggattgacaaccagctgcgcggacgttcaggccgtcagggggaccctggtatcacccaattttatctgtccatggaagatgaattaatgaaacgcttcggcgcagagcggacgatggcgatgcttgaccgcttcggaatggacgattcgacgccgatacagagcaagatggtttcaagagcggtcgaatcttcacagaagcgtgtggaaggcaacaactttgatgcccgtaagcagcttctgcaatacgatgacgtgctccgccagcagcgcgaagtcatctataaacagcgctttgaggtcatcgattccgataacctccgctccatcgtcgaaaatatgattaaagcttcactcgagcgggctgttgcttcatatacgccgaaggaagatctgcctgaagagtggaatcttgacggccttgtggagcttgtaaatgcgaatttccttgatgaaggtggagtggagaaaagcgacattttcggaaaagagcccgaggagattacagagctcatttacgaccgcatcaaaacgaaatacgatgagaaagaagagcggtacggctctgaacaaatgcgcgaatttgagaaagtcatcgttctccgcgaagtggatacgaaatggatggatcacatcgatgcgatggatcagctgcggcaaggaattcatctgcgcgcttatgctcagacaaacccgctccgcgagtatcagatggaaggctttgcaatgtttgaaaacatgatcgcggcgattgaagatgatgtagccaaattcgtcatgaaggctgaaatcgaaaacaaccttgagcgcgaagaggtcattcaaggacagacgacagcccatcagccgaaagaaggcgatgaggaaaaacaagcgaagaaaaaaccggtccgcaaagcggtggatatcggacgcaatgatccttgctactgcggaagcggaaaaaaatataaaaactgctgcggaagaacagaataaaaagaggtgcacgcctctttttatttg

SEQ ID NO: 6 - protein, 841 aa, Bacillus licheniformis
MLGILNKVFDPTKRTLSRYEKKANEIDALKADIEKLSDEALKQKTIEFKERLEKGETVDDLLVEAFAVVREASRRVTGMFPFKVQLMGGVALHEGNIAEMKTGEGKTLTSTMPVYLNALSGKGVHVVTVNEYLASRDAEEMGKIFEFLGLTVGLNLNSLSKDEKREAYAADITYSTNNELGFDYLRDNMVLYKEQMVQRPLHFAVIDEVDSILIDEARTPLIISGQAAKSTKLYVQANAFVRTLKADQDYTYDVKTKGVQLTEEGMTKAEKAFGIENLFDVRHVALNHHIAQALKAHAAMHKDVDYVVEDGQVVIVDSFTGRLMKGRRYSDGLHQAIEAKEGLEIQNESMTLATITFQNYFRMYEKLAGMTGTAKTEEEEFRNIYNMQVVTIPTNKPIARDDRPDLIYRTMEGKFKAVAEDVAQRYMVGQPVLVGTVAVETSELISRLLKNKGIPHQVLNAKNHEREAQIIEDAGQKGAVTIATNMAGRGTDIKLGEGVKELGGLAVIGTERHESRRIDNQLRGRSGRQGDPGITQFYLSMEDELMKRFGAERTMAMLDRFGMDDSTPIQSKMVSRAVESSQKRVEGNNFDARKQLLQYDDVLRQQREVIYKQRFEVIDSDNLRSIVENMIKASLERAVASYTPKEDLPEEWNLDGLVELVNANFLDEGGVEKSDIFGKEPEEITELIYDRIKTKYDEKEERYGSEQMREFEKVIVLREVDTKWMDHIDAMDQLRQGIHLRAYAQTNPLREYQMEGFAMFENMIAAIEDDVAKFVMKAEIENNLEREEVIQGQTTAHQPKEGDEEKQAKKKPVRKAVDIGRNDPCYCGSGKKYKNCCGRTE